(19) United States
(12) Patent Application Publication
(10) Pub. No.: US 2015/0307546 A1
(43) Pub. Date: Oct. 29, 2015

(54) THERMO-RESPONSIVE LECTIN-ELP FUSION BINDING LIGANDS FOR GLYCOPROTEIN PURIFICATION BY AFFINITY PRECIPITATION

(71) Applicant: GEORGIA TECH RESEARCH CORPORATION, Atlanta, GA (US)

(72) Inventors: Rachel Ruizhen Chen, Atlanta, GA (US); Lindsay Grace Arnold, Atlanta, GA (US)

(21) Appl. No.: 14/699,203

(22) Filed: Apr. 29, 2015

Related U.S. Application Data
(60) Provisional application No. 61/985,491, filed on Apr. 29, 2014.

(51) Int. Cl. C07K 1/32; C07K 14/47; C12N 9/08; C07K 14/28; C07K 14/195; C07K 14/00

(52) U.S. Cl. CPC C07K 1/32; C07K 14/195; C07K 14/00; C12N 9/0065; C07K 14/28; C07K 14/47; C07K 2319/00; C12Y 111/01007

(57) ABSTRACT

The invention provides novel affinity fusion ligands, and method of use thereof, for glycoprotein purification by affinity precipitation. The affinity fusion ligand of the invention comprises a bacterial lectin, e.g., from Ralstonia solanacearum (RSL) that binds to fucose, and/or from the Vibrio cholera neuraminidase (VCNA) that binds to saialic acid, fused with a thermo-responsive polypeptide, e.g., an elastin-like polypeptides (ELP) repeats. Methods of using the lectin-ELP fusion ligand for purifying a glycoprotein via affinity precipitation are also provided herewith.

### CROSS-REFERENCE TO RELATED APPLICATIONS

This application claims priority to, and the benefit under 35 U.S.C. §119(e), of U.S. Provisional Patent Application No. 61/985,491, filed Apr. 29, 2014, the entire contents and substance of which are hereby incorporated by reference as if fully set forth below.

### STATEMENT AS TO FEDERALLY SPONSORED RESEARCH

This invention was made with a government support under Grant No. CBET0965973 awarded by the National Science Foundation. The government has certain rights in this invention.

### FIELD OF THE INVENTION

The invention is directed to thermo-responsive fusion binding ligands comprising lectin fused with elastin-like polypeptide (ELP), and methods of use thereof, for purifying glycoprotein or glycoconjugates by affinity precipitation.

### BACKGROUND OF THE INVENTION

The current state-of-art technology for the purification of recombinant proteins is affinity chromatography (Floss et al., 2010). While effective in most cases, the costs associated with affinity ligand and column operation are high. The area of downstream pharmaceutical purification stands to receive the greatest financial benefit with the development of an affinity chromatography alternative. Over 80% of the cost associated with therapeutic protein production is found in the downstream processing. Despite a projected 48% general expansion of mammalian and microbial product production, the improvements in downstream processing are incremental (Lightfoot & Cockrem, 2013). Standard, popular methods of purification often include scaled up versions of successful bench-top methods, such as affinity chromatography. However, these methods often succumb to certain drawbacks, such as flow rate limitation, diffusion constraints, and high maintenance costs related to column fouling or ligand leaching (Jungbauer, 1993; Patchornik & Albeck, 2005).

Recently, a technique that make use of elastin-like polypeptides (ELPs) has shown promise in providing an affinity purification procedure without the costly column operation (Banki et al., 2005; Chow et al., 2006; Hilbrig et al., 2006; Hilbrig and Freitag, 2003; Meyer et al., 2001; Meyer and Chilkoti, 1999; Simnick et al., 2007). This is owing to the temperature-dependent, reversible self-aggregation (also known as inverse phase transition) of ELPs. ELPs are soluble below the transition temperature (Tt) and become insoluble above Tt. When fused to a target protein, the thermal-responsive phase transition remains intact, providing a convenient method for isolating the target protein-ELP fusion by a simple change of temperature. This method has been shown effective for ELP-fusions produced from E. coli expression systems as well as higher cells such as plant cell culture (Conley et al., 2009; Tian and Sun, 2011) and mammalian cell cultures and for proteins from small to complex such as antibody (Madan et al., 2013), suggesting its broad applicability.

Regarding purification of glycoproteins, while fusing ELP to a glycoprotein was previously shown to be successful in purifying the fusion protein with a respectful yield of 141 μg/g tobacco leaf (Lin et al., 2006), the purification requires a fusion of a target glycoprotein with an ELP sequence of at least 100 amino acids.

Glycosylation is the most prevailing post-translation modification event and impacts on a wide range of biological activities, including cell growth, differentiation, immune response, biological signaling, and fertilization (Varki et al 2009; Ghazarian et al 2011; Wiederschain 2013). Besides their significance to fundamental research, glycoproteins represent more than 70% of therapeutic drugs on the market, a sector of biopharmaceuticals with growth potentials in many therapeutic areas, including cancer treatment (Ekins and Xu 2009). Animal or plant derived glycoproteins are also widely used as therapeutics, diagnostics, and as research reagents (Nakano et al 2006; Jefferis 2009). All these applications require efficient and cost effective methods for isolation and purification of glycoproteins. However, obtaining homogenous glycoproteins is particularly challenging due to the high concentrations of other protein contaminants, low abundance of target glycoproteins, and heterogeneity in glycosylation (Taniguchi et al 2001; Raman et al 2006; Gabius et al 2011).

Lectin affinity chromatography has the potential for a one-step purification of glycoproteins. Many lectins have been identified as potential ligands for targeting glycoproteins (Fraguas et al., 2004). However, as a method of glycoprotein purification, it has many challenges. Most frequently cited are: a) high operation and maintenance costs, including the cost of affinity ligand. b) ligand binding capacity and flow rate limitations resulting in large dilution of product stream c.) column fouling which requires harsh sanitizing conditions that lead to ligand leaching, product degradation and byproduct formation (Jungbauer 1993; Patchornik and Albeck 2005). For these reasons, lectin affinity chromatography for glycoprotein purification has been limited to small scale applications (Lam and Ng 2011; Lee et al 2012). The need to develop more cost-effective methods for protein or glycoprotein purification has motivated researchers to look for non-chromatographic methods for protein purification (Hilbrig et al 2006; Gottschalk 2010; Lee et al 2012).

Affinity precipitation is an alternative approach with the potential to overcome the challenges associated with affinity chromatography, as it retains the specific interaction of affinity ligand with the protein of interest while avoiding column operation (Linné-Larsson et al 1996). Thermo-responsive precipitation is of particular interest as it provides a convenient way to toggle between solution phase where the affinity ligand can interact with the target protein under optimal conditions, and solid phase where protein complex can be isolated from the precipitate in significantly enriched form. Elastin-like protein (ELP), as a thermal responsive biopolymer, has been used to facilitate precipitation for protein purification (Urry et al., 1992; Fong et al 2009; Madan et al 2013). However, ELP has not been used to purify glycoproteins based on glycoforms present on the protein.

Affinity precipitation is increasingly being embraced as an alternative to affinity chromatography in protein purification. Since the first demonstration by Meyer and Chilkoti (1999) of the use of ELP in affinity precipitation, many researchers have contributed to the development of the method. ELP could be used as a fusion partner to a target protein and produced as a fusion protein. After purification, ELP could be removed by protease cleavage (Meyer and Chilkoti 1999; Meyer et al 2001). Further development with intein-mediated self-cleavage eliminates the protease use in ELP removal (Banki et al., 2005; Ge et al., 2005; Tian and Sun, 2011; Wood, 2010). Recently, it was shown that, instead of fusing ELP to a target, it could be fused to an affinity ligand. Recombinant proteins are produced as a fusion with a small affinity tag recognized by the affinity ligand fused with ELP. This approach has the advantage that recombinant protein target could be produced independent of ELP and likely results in higher expression level, as ELP portion would otherwise add 200 amino acid if fused with the target protein (Shimazu et al 2003; Liu et al 2012). This approach also addresses misfolding occurring in some large ELP-target protein fusions.

Plant peroxidases are glycoproteins widely used as research reagents. With their activity easily and inexpensively detected, they are essential in numerous commercial diagnostic kits. They are also used widely in food processing and in waste water treatment (Chattopadhyay and Mazumdar 2000; VanHaandel et al 2000). Horseradish peroxidase (HRP), for example, has been used as a conjugate for CL-ELISA for detection of antibody-mediated binding events, N-de-methylation to produce food flavor, and in removal of dyes from polluted water (VanHaandel et al 2000; Husain 2009; Sakharov et al 2010). Soybean peroxidase (SBP) is functionally equivalent to HRP but more thermal and environmentally stable. As such, it can be used in applications requiring more harsh conditions than HRP can tolerate and extend shelf life of peroxidase containing diagnostic kits (Mceldoon and Dordick 1996; Henriksen et al 2001; Kamal and Behere 2002). SBP could potentially be derived from soybean hull, a byproduct of the soybean industry (Gillikin and Graham 1991; Hailu et al 2010). However, SBP exists in a complex plant crude extract with a low concentration of 3% of the total hull protein content (Hailu et al 2010). It requires an efficient isolation and purification process to achieve a suitable purity for a target application and to meet the cost objectives.

Sialic acid, a monosaccharide found mostly as a terminal sugar of a glycan, is of great physiological and pathological importance on certain therapeutics. Sialic acid, or N-acetylneuraminic acid, is a larger monosaccharide comprised of a 9 carbon backbone and an acetyl group that confers a negative charge to the molecule. Because of the importance of sialic acid residues, extensive studies of sialylated glycoproteins and their glycoforms have been researched. It has been shown that additional sialic acid residues increase the negative net charge and improve the pharmacokinetic properties of certain glycoproteins (Sethuraman & Stadheim, 2006; Misaizu et al., 1995).

There is a need for effective methods for isolation and purification of glycoproteins, such as plant peroxidases HRP and SBP, and/or sialylated proteins fetuin, and other glycoconjugates because they are important to biopharmaceutical industry and diagnostic industry, as well as to an emerging field of glycoproteomics.

### SUMMARY OF THE INVENTION

The invention provides an affinity binding ligand comprising a fusion construct comprising lectin fused with a thermal responsive elastin-like protein (ELP), wherein said affinity binding ligand is for purifying a glycoprotein by affinity precipitation. In one specific embodiment, the lectin is derived from bacteria Ralstonia solanacearum (RSL) and binds to fucose. In another specific embodiment, the lectin is derived from Vibrio cholera neuraminidase (VCNA) and binds to sialic acid. In certain embodiments, the fusion construct further comprises a 10 amino acid glycin rich linker (GGGGS)₂ to link lectin and ELP.

In certain embodiments, the ELP comprises 20, 40, or 60 ELP pentapeptide repeats of VPGVG (SEQ ID NO:1). In certain specific embodiments, the fusion construct is RSL-ELP20, RSL-ELP40, or RSL-ELP60, wherein the lectin is derived from RSL and is fused with 20, 40, or 60 VPGVG (SEQ ID NO:1) ELP repeats, respectively. In certain embodiments, these fusion constructs are used for purifying plant peroxidases, such as horseradish peroxidase (HRP) or soybean peroxidase (SBP), and/or any other glycoprotein or glycoconjugates containing a fucose moiety.

In other specific embodiments, the fusion construct is VCNA-ELP20, VCNA-ELP40, or VCNA-ELP60, wherein the lectin is derived from VCNA and is fused with 20, 40, or 60 VPGVG (SEQ ID NO:1) ELP repeats, respectively. In certain embodiments, these fusion constructs are used for purifying sialylated proteins, such as fetuin, and/or any other glycoprotein or glycoconjugates containing sialic acid portion.

The invention further provides a method for purifying a glycoprotein of interest by affinity precipitation comprising the steps of a) constructing a fusion binding ligand comprising lectin fused with ELP pentapeptide repeats of VPGVG (SEQ ID NO:1); b) contacting the fusion binding ligand with said glycoprotein of interest; and c) isolating and purifying said glycoprotein of interest by affinity precipitation. In certain embodiments, the lectin of the fusion binding ligand is derived from bacteria and binds to fucose, sailic acid, or a glycan portion of the glycoprotein of interest. In certain embodiments, the invention method is successfully used for purifying the plant peroxidase, such as HRP and SBP, as well as for purifying the sialylated protein, such as fetuin, by affinity precipitations. Further, the glycoprotein of interest is isolated and purified with Inverse Temperature Cycling (ITC) in the invention method.

These and other aspects of the present invention will be apparent to those of ordinary skill in the art in the following description, claims and drawings.

### BRIEF DESCRIPTION OF THE DRAWINGS

This patent application file contains at least one drawing executed in color. Copies of this patent application with color drawing(s) will be provided by the Office upon request and payment of the necessary fee.

FIGS. 1A-1D. FIG. 1A: SDS-PAGE of extracellular milieu after 48 hours. Lane M: Molecular weight marker; Lane 1: E609Y empty vector control; Lane 2: E609Y expressing RSL protein; Lane 3: E609Y expressing RSL-ELP20 protein; Lane 4: E609Y expressing RSL-ELP40 protein; Lane 5: RSL-ELP40 purified by one cycle of ITC; Lane 6: RSL-ELP20 purified by one cycle of ITC. FIG. 1B: SDS-PAGE of RSL-ELP20 purified by fucose affinity chromatography. Lane M: Molecular weight marker; Lane 1: Crude protein extract from E609Y with RSL-ELP20 monomer seen at 25 kDa; Lane 2: RSL-ELP20 in dimer and trimer in eluent with 0.1M fucose. FIG. 1C: SDS-PAGE of HRP purified from an E. coli lysate. Lane 1: E. coli lysate protein with HRP; Lane M: Molecular weight marker; Lane 2: Supernatant after RSL-ELP40 addition and salt mediated aggregation at room temperature; Lane 3: Resuspended pellet (containing RSL-ELP40 and HRP complex); Lane M2: Molecular weight marker; Lane 4: Isolated HRP after removal of RSL-ELP by ITC. FIG. 1D: SDS-PAGE of fetuin experiment. Lane 1: Resuspended RSL-ELP40 pellet with no visible fetuin; Lane 2: Supernatant after RSL-ELP40 addition and salt mediated aggregation at room temperature; Lane 3: Resuspended RSL-ELP40 trimerized by addition of fucose with no visible fetuin; Lane M: Molecular weight marker.

FIG. 2: Recovery yield as a function of increasing molar ratio of RSL-ELP40 to HRP.

FIG. 3: Lane 1. Crude soybean hull protein solution. Lane 2. Purified soybean peroxidase. Lane 3. Marker. Lane 4. Isolated RSL-ELP40 after SBP purification.

FIG. 4: Activity and recovery yield of SBP as a function of the ratio of RSL-ELP to SBP.

FIG. 5: Turbidity of RSL-ELP with increasing temperature.

FIG. 6: Activity in the bound and unbound fractions when RSL-ELP40 bound SBP complex is suspended in elution buffers of varying fucose concentration.

FIGS. 7A-7B. FIG. 7A: Recovery activity, isolation activity, and activity left in wash over the course of several recycles. FIG. 7B: Distribution of measured activity in each fraction.

FIG. 8. Turbidity profile to determine transition temperature of VCNA-ELP40.

FIG. 9. Lane 1: Fetuin stock solution; Lane 2: Uncaptured fetuin after VCNA purification; Lane 3: Isolated VCNA after purification; Lane 4: MW ladder; Lane 5: Isolated fetuin.

FIG. 10. Recovery of fetuin as a function of ligand to target ratio.

FIG. 11. Purified fetuin from CHO cell media.

FIG. 12. Purified Fetuin from high albumin solutions.

FIG. 13. Isolation yield and % albumin depleted as a function of initial albumin concentration (Fetuin).

FIG. 14. Purified HRP from high albumin solutions.

FIG. 15. Isolation yield and % albumin depleted as a function of initial albumin concentration (HRP).

### DETAILED DESCRIPTION OF THE INVENTION

The invention provides a novel affinity precipitation method for glycoconjugate and/or glycoprotein purification using a designed novel thermo-responsive affinity binding fusion ligand comprising lectin and ELP.

As used herein, the term “glycoconjugate” is the general classification for carbohydrates covalently linked with other chemical species such as proteins, peptides, lipids and saccharides. Glycoconjugates are formed in processes termed glycosylation. As used herein, the term “glycoprotein” refers to a protein that contains oligosaccharide chains (glycans) covalently attached to polypeptide side-chains. The invention method contemplates the broadest scope for purification of any glyco- and/or glycan-linked molecules by affinity precipitation.

As used herein, the term “lectin” refers to carbohydrate-binding proteins, macromolecules that are highly specific for sugar moieties. A comprehensive list of lectins: origins, natures, and carbohydrate specificities is provided by Kobayashi et al (Methods Mol Biol. 2014). The invention method contemplates the broadest scope for constructing the fusion binding ligand comprising any lectin derived from various origins and natures, as long as its carbohydrate specificities make it to bind specifically to the glycan portion of the glycoprotein or glycoconjugate of interest.

As used herein, the term “ELP” refers to peptide polymers that are composed of repeats of the pentapeptide (Val-Pro-Gly-Xaa-Gly)n where Xaa is the guest residue; this residue can be any amino acid except Pro; n describes the number of repeats and is typically between 20-330 (Meyer and Chilkoti 2004). ELPs undergo a sharp and reversible phase transition when heated above their lower critical solution temperature (LCST). ELPs retain this behavior when they are fused to a protein, and thereby provide a simple method to isolate a recombinant ELP fusion protein from cell contaminants by cycling the solution through the insoluble and soluble phase of the ELP fusion protein by a method termed Inverse Transition Cycling (ITC). This method does not require the use of chromatography, so that it is cost effective, easy to scale up (Hassouneh et al. 2010).

Taking advantage of the small size of bacterial lectins, the invention provides the lectin-ELP fusions as affinity ligand. The invention further provides that these fusion affinity ligands, as demonstrated herewith, could be used to purify glycoproteins based on the glycoforms present on a protein surface. This sets apart the work from previous other works that are all based on the affinity interaction with polypeptide backbone. This difference is important as glycans attached to a protein are often functionally significant. Therefore, the glycan-specific approach not only extends useful ELP-based affinity precipitation to a large class of glycoproteins, but also provides a new non-chromatography purification mechanism based on the affinity interaction between carbohydrates and protein ligand, naturally present but not exploited to its fullest for purification. Since there is no tag on a target protein, the glycoprotein isolated from the invention method is authentic and no post-purification processing is needed. Besides useful in recombinant glycoprotein purification, the invention method also opens up vast opportunity to purify glycoproteins from natural plant and animal sources, two major sources of diagnostic enzymes and research regents.

In certain embodiments, the designed novel thermo-responsive sugar binding ligand is a fusion affinity ligand comprising a bacterial fucose lectin fused with a thermo-responsive polypeptide. In certain embodiments, the novel thermo-responsive sugar binding ligands were designed by fusing an ELP sequence with a small bacterial lectin. In certain embodiments, the bacterial lectin is from Ralstonia solanacearum (RSL), and fusion constructs comprises RSL-ELP20, RSL-ELP40, and/or RSL-ELP60. The ability of lectin-mediated glycan recognition and affinity binding, combined with the reversible phase transition of ELP, allows a new method of glycoprotein purification on the basis of carbohydrate present on the glycoprotein.

In one specific embodiment, the invention described herewith is the first application of the invention new purification method using a fucose lectin and horseradish peroxidase (HRP) as model lectin and target glycoprotein, respectively. In certain embodiments, the invention method features an integrated process for production of the designed thermal responsive affinity ligands by using an E. coli system capable of extracellular secretion of recombinant proteins, and a high yield (95%) of recovery of these designer affinity ligands directly from growth medium by using only Inverse Temperature Cycling (ITC). The purification process with a fucosylated protein, was efficient, achieving high recovery yield. The affinity purification process was also specific, working well even with large amounts of cellular debris and high concentration of other glycoproteins. Additionally, the invention method developed was sensitive and able to fully recover a low abundant glycoproteins. By developing other sugar binders in the similar fashion, the invention method can be highly useful for glycoprotein purification and detection.

The invention further provides that the lectin-mediated affinity precipitation could be used to purify a useful and valuable enzyme, e.g., soybean peroxidase (SBP), a fucosylated protein, from a dilute and complex plant crude extract. In certain embodiments, the invention provides that the capture of SBP relies on the binding of fucose on SBP to the lectin in the RSL-ELP40 ligand. It was found that the recovery yield, a measure of capture efficiency, has a strong dependence on the molar ratio of ligand to target protein.

In one specific embodiment, an affinity precipitation process was developed and used for purifying a low abundant SBP from a complex plant crude extract. Under optimal conditions, one step binding and precipitation resulted in >95% recovery yield directly from crude extract and a 22.7-fold purification. The product isolated using this affinity precipitation meets or exceeds the quality specifications of comparable products by Sigma. This process showed that the recovery yield had a strong dependence on the molar ratio of ligand to the target fucosylated protein, with a ratio of three being optimal for SBP, which could be predicted based on the total fucose content per protein molecule and the number of binding site per ligand molecule. The optimal condition for elution of target protein was determined and 1 mM fucose resulted in an isolation yield (80%). The demonstrated recyclability of ligands provides opportunities to reduce the cost of affinity ligand to further ensure the cost-effectiveness of the bioseparation process.

The invention further provides a new sialic acid binding lectin as a fusion construct for affinity precipitation purification of relevant therapeutic glycoproteins of clinical interest. In one specific embodiment, the N-terminal of lectin from Vibrio cholera neuraminidase (VCNA) is efficient in targeting terminal sialic acid moieties and does not require the addition of metal ions for binding, while the C-terminal of lectin is fused to a glycine rich linker connecting the gene encoding the (VPGVG)₄₀ sequence of the ELP domain. In certain embodiments, the invention provides that the fusion construct, e.g., VCNA-ELP40, can be used for purifying a plant peroxidase, e.g., HRP and SBP, and a sialylated protein, such as fetuin.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Novel Thermo-Responsive Fucose Binding Ligands for Glycoprotein Purification by Affinity Precipitation

This example describes novel thermo-responsive affinity sugar binders that were developed by fusing a bacterial fucose lectin with a thermo-responsive polypeptide. These designer affinity ligands fusions were produced using an E. coli system capable of extracellular secretion of recombinant proteins and were isolated with a high recovery yield (95%) directly from growth medium by Inverse Temperature Cycling (ITC). With horse radish peroxidase (HRP) as a model protein, this example demonstrates that the designer thermo-responsive ligands are capable of interacting with glycans on a glycoprotein, a property that was used to develop a novel affinity precipitation method for glycoprotein purification. The invention method, requiring only simple process steps, affords full recovery of a target glycoprotein, and is effective at a very low target glycoprotein concentration in the presence of large amounts of contaminants. By developing other sugar binders in the similar fashion, the method could be highly useful for glycoprotein purification and detection.

To avoid a fusion to target glycoprotein, which may require post-purification cleavage for authentic proteins, novel thermo-responsive sugar binding ligands were designed by fusing an ELP sequence with a small bacterial lectin. The ability of lectin-mediated glycan recognition and affinity binding, combined with the reversible phase transition of ELP, allows a new method of glycoprotein purification on the basis of carbohydrate present on the glycoprotein. Described herewith is the first application of this new purification scheme using a fucose lectin and horseradish peroxidase (HRP) as model lectin and target glycoprotein, respectively.

Design and Production of Lectin-ELP Fusions.

Lectins derived from plants are used widely in identifying carbohydrates. They are generally large multimers, not readily functionalized by genetic means. To develop ELP-lectin fusions, bacterial lectins with much smaller sizes are better suited. Additionally, their bacterial origins are much more conducive to be expressed by bacterial systems in a cost effective manner. As a model lectin, a fucose lectin from Ralstonia solanacearum (RSL), which is a small protein with its monomer size of 9.9 kDa, was selected. Constructs of 20, 40, and 60 ELP pentapeptide repeats were designed in order to identify the best configuration of ELP-lectin fusion for the application. In order to develop cost-effective method for production of these affinity ligands, the fusions were expressed with a TAT signal sequence in an E. coli system and extracellular secretion was achieved by using the method previously developed in Chen Lab (Shin and Chen, 2008). Each confirmed plasmid was transformed into E. coli E609Y, carrying a deletion of the lpp encoded lipoprotein, resulting in significant increase in outer membrane permeability to allow periplasmic proteins to leak into growth medium (Chen, 2007; Shin and Chen, 2008).

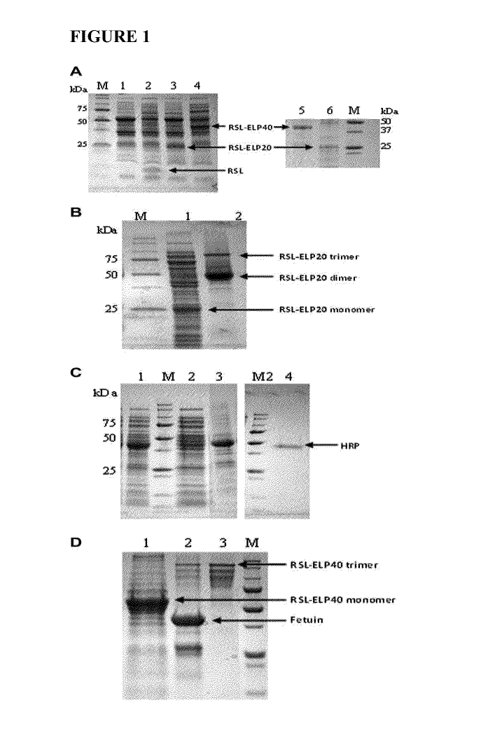

E609Y transformants were grown in LB medium and extracellular proteins were collected to monitor the secretion of recombinant fusion. As shown in FIG. 1A, the two recombinant lectin-ELP-fusions, RSL-ELP20 and RSL-ELP40, were easily identified from a SDS-PAGE gel as a significant band. Based on the band intensity, protein yield was estimated and shown in Table 1. In general, the yield of recombinant proteins decreases with the increase of fusion size. The highest protein yield was obtained with the naked lectin, at 35 mg/L. For the largest construct, lectin fused with 60 repeats of pentapeptide, no extracellular fusion was found; instead, the protein appeared to accumulate in the periplasm, with its periplasmic concentration being the highest among all constructs.

TABLE 1
Total recombinant protein produced after 48 hours.

| | Extracellular | Periplasmic |
|---|---|---|
| Construct | (mg/L) | (mg/L) |
| RSL | 35 ± 7 | 2.8 ± .6 |
| RSL-ELP20 | 30 ± 6 | 1.4 ± .3 |
| RSL-ELP40 | 18 ± 4 | 1.6 ± .3 |
| RSL-ELP60 | 0 ± 0 | 5.7 ± 1.1 |

The extracellular secretion makes it possible to harvest proteins without cell breakage or addition of protease inhibitors. Taking advantage of the reversible phase transition afforded by the ELP functionality, the recombinant protein fusion can be purified by one or two cycles of temperature induced phase transition. As shown, after one cycle of inverse temperature cycling (ITC), fusion proteins could be obtained in sufficiently pure form (FIG. 1A) for the target application. Recovery yield of recombinant protein is typically 70-80% with one cycle of ITC and 95% with two cycles of ITC. This feature helps to reduce the cost in production of affinity ligand for the application of interest.

Recombinant Fusion Retains Dual Functionality.

By design, these fusion proteins are bifunctional. To test the functionality of lectin of the fusion, the binding ability of RSL was tested using a Fucose Separopore® (Agarose) matrix. The unbound fraction was washed from the column with PBS, and the bound fraction was eluted from the column. As seen in FIG. 1B, after elution with fucose, lectin-ELP dimer and timer were formed. It was previously reported that trimer was the active form of the lectin (Kostlanova et al., 2005). However, the observation of trimer and dimers of the fusion (FIG. 1B) indicates that multimerization of the lectin is not interrupted by the fusion. In fact, the association of dimer and trimer form in the presence of fucose is strong enough to withstand the harsh SDS-PAGE sample preparation procedures. Only after exhaustive washing with buffer, the monomer was observed in a SDS-PAGE. These observations indicate that the lectin functionality is retained within the fusion. The functionality of the ELP tag was tested by observing reversible phase transition. By following reported methods (Christensen et al., 2009; Meyer et al., 2001; Shimazu et al., 2003), the transition temperature was determined to be 80, 45, and 38° C., for RSL-ELP20, RSL-ELP40, and RSL-ELP60, respectively. These transition temperatures are close to reported values for the same size of ELP, either alone or fused with a protein (McDaniel et al, 2013; Christensen et al, 2013). They also match well with model predicated values (Meyer et. al., 2004). For example, the transition temperature of the ELP fusion used was predicted to be 44° C. and the measured transition temperature was 45° C. The salt effect on transition temperature was also investigated. It was observed that the transition temperature for RSL-ELP40 was reduced to 36° C. and room temperature in the presence of 0.5M NaCl and 1 M NaCl, respectively. The aggregation process was fully reversible and behaved similarly over the course of multiple cycles of temperature ramping and cooling. Taken together, these results indicate the fusion retains both functions. Considering both production and transition temperature, RSL-ELP40 is particularly suitable as affinity ligand for glycoprotein purification, with reasonable extracellular production yield and transition temperature low enough to avoid denaturing of target proteins.

Application in Affinity Purification of a Horseradish Peroxidase.

To demonstrate the utility of the designed ligand for affinity purification of glycoprotein, a fucose containing glycoprotein, horseradish peroxidase (HRP), was used as the model glycoprotein. HRP, a 441(D protein, is core-fucosylated at eight out of nine glycosylation sites (Wuhrer et al., 2005). As a first test, RSL-ELP40 (1.0 mg, 0.2 mM) was mixed with HRP (0.1 mg, 0.01 mM) in a PBS solution pH 7.4 in a 1.5 ml Eppendorf tube. After overnight incubation on ice with shaking at 60 RPM overnight, the mixture was brought to room temperature, and NaCl was added to a final concentration of 1.0 M. Precipitation was observed immediately and allowed to complete at room temperature for 20 minutes, upon which the precipitates were spun down by centrifugation at 15,000 g for 20 minutes. The pellet was removed from supernatant and re-solubilized by resuspension in PBS buffer solution at room temperature. Samples of the supernatant and resuspended pellet were taken for residual and isolated peroxidase activity, respectively. It was found that using room temperature for precipitation, there was no reduction of activity during ITC. Under the condition, the recovery yield of HRP was 98% (±2.8%) based on activity assay. Finally, purified HRP was isolated from RSL-ELP40 by an additional round of ITC. To this end, the pellet of complex of HRP and RSL-ELP40 was resuspended in PBS buffer, and 0.1M fucose was added. The addition of fucose released HRP from its complex with lectin, and then the ligand, RSL-ELP40, was collected by precipitation with 1.0 M NaCl, leaving HRP as the only protein in the solution.

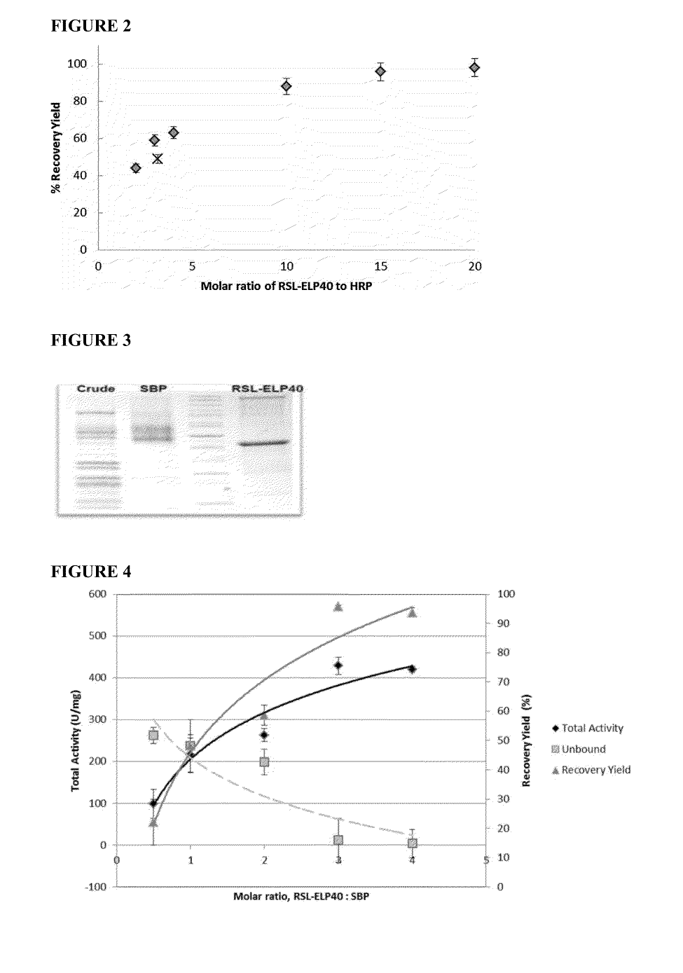

To investigate a relationship between recovery yield and the amount of ligand used in the purification process, the molar ratio of ligand to HRP was varied from 1:2 to 1:20. Results showed that recovery yield increased with the increase of ligand relative to the amount of target protein present in the mixture and full recovery was approached with 15-20 molar excess of ligand (FIG. 2). Under the excess ligand condition, the lowest target protein concentration that was recovered with 100% yield (based on activity assay) was 1.4 pM HRP, indicating that this method of protein recovery is highly sensitive.

To investigate how cellular protein and other debris affect purification, E. coli whole cell lysate with protein concentration 10 mg/ml was mixed with target protein 0.25 mg/ml (5.7 μM) HRP. RSL-ELP40 fusions were added to a final concentration of 0.5 mg/ml (18 μM). After one cycle of ITC, followed by addition of fucose to precipitate ligand, HRP was obtained in highly pure form, shown as a single band (FIG. 1C), suggesting that the quality of purified protein maintained even in the presence of large amount of protein contaminants and other cellular debris. The recovery yield in this case was 49±0.9%, slightly lower than the case without contaminating debris (FIG. 2), suggesting no major impact on recovery yield by contaminants, illustrating the advantage of the affinity purification process. Even at very low target protein concentration, the presence of cell lysate reduced the recovery of HRP only slightly, from 98% (±2.8%) to 90±8.6%.

To further illustrate that the purification is specific for fucose-containing glycoproteins, an experiment was conducted with fetuin used as a glycoprotein target. Fetuin is a non-fucose containing glycoprotein of size 48 kDa isolated from fetal calf serum (Spiro, 1960). The experiment was carried out as described above except the target protein is fetuin. As shown in FIG. 1D, fetuin appeared only in the soluble fraction after ITC (lane 2), and there was no fetuin present in re-solubilized pellet (lane 3), indicating its inability to form complex with RSL-ELP40. Another experiment was conducted with the glycoprotein mixture containing an equimolar ratio (0.1 M) of HRP and fetuin, HRP was recovered with no reduction of yield when compared to the case without fetuin addition (not shown). These results indicate that the presence of a non-fucose-containing glycoprotein did not interfere with the purification and the affinity precipitation is sufficiently specific to fucose containing glycoprotein.

### Materials and Methods

Chemicals.

All chemicals were purchased from Sigma-Aldrich Chemical Co. (St. Louis, Mo.) unless otherwise specified and were used without further purification.

Bacterial Strains and Plasmids.

Escherichia coli strain K12 UT5600 (F-ara-14 leuB6 secA6 lacY1 proC14 tsx-67 Δ(ompT-fepC)266 entA403 trpE38 rfbD1 rpsL109 xyl-5 mtl-1 thi-1) was used as a host for all cloning steps. Plasmid pQTAT containing an N-terminal TAT signal sequence was used as the expression vector. The artificial gene (encoding the lectin, a 10 amino acid glycine rich linker and 20 VPGVG repeats) was synthesized by GenScript. The ELP segment with 20 repeats of pentapeptide was oligomerized to create 40 and 60 pentamer ELP, respectively, which was subsequently fused with the same linker and lectin in the same manner as the first construct. The synthetic genes and the expression vector pQTH, were digested with BamHI and HindIII restriction enzymes (NEB), and ligated to form the respective plasmids for fusion expression.

Protein Expression and Fractionation.

A single colony of E. coli 609Y transformant was selected and cultured in 3 mL LB broth and ampicillin overnight at 37° C. and 250 RPM. This seed culture was used to inoculate 100 mL LB broth culture containing ampicillin to an OD₆₀₀ of 0.1. This larger culture was grown at 37° C. and 250 RPM to a cell density of OD₆₀₀ of about 0.4, at which point isopropyl-β-D-thio-galactoside (IPTG) was added to the culture to a final concentration of 1 mM to induce the production of the fusion protein. The cultivation temperature was then reduced to 18° C. and the cells were grown for an additional 48 hours.

The extracellular fraction was collected as the supernatant after pelleting the cells of 10 mL of culture media by centrifugation at 6,000 g at 4° C. for 25 min. This was repeated until supernatant was clear. Periplasmic and cytoplasmic fractions were prepared according to the method described in the pET system manual (EMD Chemicals, San Diego, Calif.). A ten milliliter culture was harvested by centrifugation and re-suspended in 5 ml of shock buffer solution comprised of 30 mM Tris-HCl, pH 8.0, 20% sucrose, and 1 mM EDTA. The cell suspension was incubated at room temperature for 10 min and pelleted by centrifugation at 6,000 g at 4° C. for 10 min. The cells were re-suspended in 0.5 ml ice-cold 5 mM MgSO4 and incubated on ice for 10 min. After centrifugation (under the same conditions as above), the supernatant was collected as a periplasmic fraction. The pellet was re-suspended in 1 ml phosphate buffered saline (PBS) solution, sonicated, and centrifuged at 6,000 g at 4° C. for 20 min. The supernatant was saved as a cytoplasmic fraction.

Isolation with Affinity Chromatography.

Fucose binding functionality within the fusions were tested with a Fucose Separopore® (Agarose) matrix, obtained from bio-WORLD (Dublin, Ohio, USA). The crude protein solution was added to this matrix and equilibrated with PBS. This mixture was kept on ice on an orbital shaker at 60 RPM overnight to allow for binding. The unbound fraction was washed from the column with PBS, and the bound fraction was eluted from the column with an elution buffer of 0.1M fucose in PBS. Samples from each stage of the process were collected and analyzed with SDS-PAGE.

Inverse Phase Transition.

The transition temperature of the constructs was determined spectrophotometrically in a 96-well microplate reader (Spectramax M5, Molecular Devices). Each well contained 200 microliters of PBS with 0, 0.5, or 1.0 M NaCl with construct at a concentration of 5 mg/mL. Temperature was increased (in 2° C. increments) from 30 to 50° C. and the absorbance at 310 nm was measured.

SDS-PAGE Analysis.

Each sample was combined in a 1:1 ratio with SDS sample buffer (10% SDS, 10% b-mercaptoethanol, 0.3 M Tris-HCl (pH 6.8), 0.05% bromophenol blue, 50% glycerol), boiled for 5 min, and resolved by 12.5% (w/v) SDS-PAGE. Each gel was detected by Coomassie blue staining (Bio-Rad).

Peroxidase Assay.

For peroxidase assay, a solution of 0.0017 M hydrogen peroxide was prepared by diluting 1 mL of 30% hydrogen peroxide to 100 mL with DI water. 1 mL of this solution was further diluted into 50 mL of 0.2 M potassium phosphate buffer pH 7.0. A 0.0025 M 4-aminoantipyrine solution was prepared in 0.17 M phenol solution. The hydrogen peroxide solution and aminoantipyrine solution were mixed 1:1 ratio, and diluted horseradish peroxidase was added. Absorbance of the resulting mixture was measured at 510 nm over a 5 minute time period at 25° C. in a microplate reader, Spectramax M5 (Molecular Devices).

### Example 2

### One-Step Non-Chromatography Purification of a Low Abundant Fucosylated Protein from Complex Plant Crude Extract

Novel thermo-responsive sugar-binding ligands were developed by fusing a small bacterial fucose lectin with an ELP (See EXAMPLE 1 above). In this Example, the fucose-binding ligand was applied to isolate SBP, a fucosylated protein, from complex plant crude extract. This example demonstrates that affinity precipitation is particularly effective in purifying a low abundant protein from a complex mixture, resulting in >95% recovery yield and 22.7-fold purification in one step. The issue of affinity ligand regeneration and its reuse in the purification process were also addressed.

In this example, the newly-developed affinity ligand, a fusion protein of elastic like polymer (ELP) and a bacterial lectin, was applied in an affinity precipitation process to purify soybean peroxidase (SBP) based on the presence of fucose on the protein surface, and the challenge of purifying a low abundant protein from a complex dilute crude plant extract was particularly addressed. The novel affinity precipitation developed in this example was very promising. One step binding and precipitation resulted in >95% recovery yield directly from crude extract and a 22.7-fold purification, giving a specific activity of 420 U/mg. The SBP isolated using this affinity precipitation meets or exceeds the quality specifications of reagent grade products by Sigma. The study showed that the recovery yield had a strong dependence on the molar ratio of ligand to target fucosylated protein, with a ratio of three giving nearly full recovery, which could be predicted based on the total fucose content per protein molecule and the number of binding site per ligand molecule. A method of ligand regeneration was also developed and its reuse was investigated. A simple wash with pH buffer was shown to be effective to regenerate the binding capacity for the ligand, and the ligand could be used for 10 times, giving an averaged 80% isolation yield based on initial input of soybean peroxidase.

Taken together, an effective method of affinity precipitation was developed, which could be used to enrich a low abundant target glycoprotein from a complex mixture with a high recovery yield. The high selectivity for fucosylated protein and its ease of operation make this method particularly useful for purification of low abundant glycoprotein from natural sources. This work establishes a non-chromatography glycoform-specific purification method and extends the useful ELP-based affinity precipitation to glycoproteins.

SBP in Soybean Crude Plant Extract.

The simple processing method of soybean hull, as described in Materials and Methods section below, resulted in a crude plant extract, in which SBP was a minor component. The total soluble protein content in the soybean crude extract is approximately 3.8 mg/ml. An SDS-PAGE analysis shows that several bands are present (FIG. 3). Based on previous studies (Hailu et al 2010), soybean peroxidase constitutes only 3% of total protein in the crude extract, thus it is not expected to appear as one distinct band. However, the presence of soybean peroxidase was indicated by its activity measured at 18.5+/−0.7 U/mg.

Crude protein samples were also analyzed for presence of sugars as glycosylation are common in plants. A total sugar concentration was measured at 3.2+/−0.5 mg/ml, which is comparable to the total protein concentration (3.8 mg/ml), suggesting significant glycosylation in plant crude sample. The averaged molar ratio of monosaccharide to the two N-Acetylglucosamine (GlcNAc) at the base of each N-glycan was shown in Table 2. Fucose to GlcNAc ratio in the crude was 1.1:2. There were also significant mannosylated proteins in the crude, with the ratio of mannose to GlcNAc 6.5 to 2. In addition, xylosylated proteins were also present. Overall, these results indicate that the targeted fucosylated protein is present at a low concentration and in a rather complex mixture containing other glycosylated proteins. It would be of interest to see how effective the affinity precipitation is in purifying a low abundant fucosylated protein from this complex plant crude extract.

TABLE 2
Molar ratios of monosaccharides
associated with crude and purified SBP

| | Molar ratios | | | | |
|---|---|---|---|---|---|
| | Fucose | GlcNAc | Xylose | Mannose | Reference |
| Crude | 1.1 ± 0.3 | 2 ± 0.2 | 1.3 ± 0.4 | 6.5 ± 0.3 | This Study |
| Purified | 1 ± 0.2 | 2 ± 0.2 | 0.9 ± 0.04 | 3.4 ± 0.3 | This Study |
| SBP | | | | | |
| Literature | 0.9 | 2 | 0.7 | 3.3 | (Gray et al. |
| SBP | | | | | 1996) |

SBP Capture by the Affinity Ligand and Precipitation of SBP-Ligand Complex.

The purification is initiated by addition of the thermo-responsive affinity ligand, RSL-ELP fusion protein, to the crude plant extract. The mixture is incubated at an ambient temperature (−25° C.) for 30 minutes to allow the lectin to bind to the fucose on the surface of SBP, resulting in formation of RSL-ELP-SBP complex. Subsequently, the mixture is brought to 50° C., and the complex precipitates out of the solution. The fraction of SBP activity captured by the ligand is denoted as recovery yield, which represents the activity captured and recovered from the crude after one round of ITC (Inverse Temperature Cycle). The fraction of SBP activity remaining in the soluble crude fraction is denoted as unbound, since the ligand failed to initially capture this fraction. Total activity refers to the absolute activity, in U/mg, of SBP targeted and isolated. As shown in FIG. 4, the recovery yield is dependent on the molar ratio of ligand to SBP. Only 20% SBP was captured by the ligand when a molar ratio of ligand to SBP of 0.5 to 1 was used. Increasing the ratio to 3 resulted in a recovery yield of 95%.

The above process was developed using 50° C. precipitation, which takes advantage of SBP's thermal stability (Sakharov et al 2010). For proteins that are less thermally stable, the transition temperature could be lowered by including salt in the precipitation step. The RSL-ELP construct was determined to have a Tₜ of 45° C. by turbidity studies (FIG. 5). Addition of 0.5M NaCl resulted in a reduction of this temperature to about 35° C., as seen in FIG. 5. Thus the method could be easily adapted to a wide range of proteins with varying stability.

Effect of Fucose Concentration on SBP Elution.

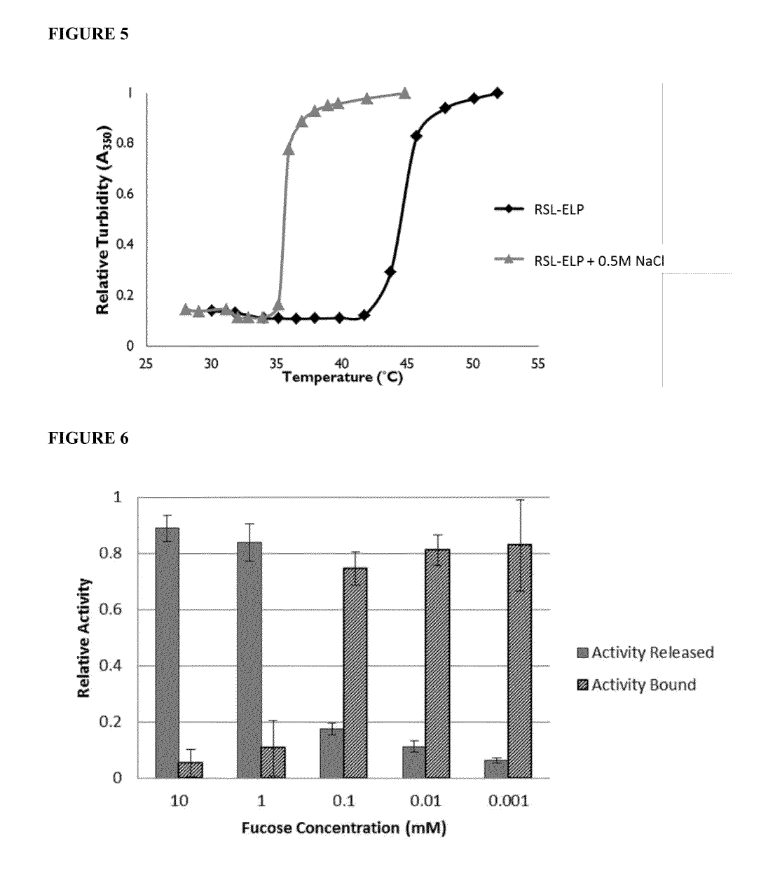

Typically, release of lectin from binding complex could be achieved by respective cognate sugar. As fucose is relatively expensive, minimizing its use is important for the SBP purification. The effect of fucose concentration on ligand release was investigated. Fucose concentration over a range of 0.001 mM to 10 mM was studied for ligand release. After one temperature cycling to precipitate ligand and bound target (as described in the last section), the complex was resuspended in an elution buffer containing the specified concentration of fucose. SBP released from the ligand was then separated from the ligand by a temperature induced precipitation. SBP activity present in the soluble portion is denoted as released activity, and was compared to the initial activity of the crude to calculate the relative released activity. SBP activity associated with the ligand is measured as the bound fraction. This bound fraction is again compared to initial activity, allowing for calculation of relative bound activity. Shown in FIG. 6 is released relative SBP activity and bound relative SBP activity as a function of fucose concentration. It appears that 1 mM of fucose is optimal, releasing about 80% SBP from protein complex, based on activity measured in the supernatant. Other methods of elution of SBP such as low pH and solvents were also tested.

Optimal Conditions for SBP Affinity Precipitation and Product Characterization.

Based on the above studies on the capture and elution of SBP, several parameters are important to the affinity precipitation process. In the capture step, they are ratio of molar ratio of ligand to target and temperature for precipitation. Using a ratio of 3 (ligand to SBP) and 50° C. for the precipitation resulted in 95% recovery yield. In the elution step, the concentration of fucose is very important and 1 mM being optimal, which gave 80% release of SBP or isolation yield. A summary of purification and the characterization of SBP isolated under the optimal conditions of this process were shown in Table 3.

In a typical experiment, after the elution step, a specific soybean peroxidase activity of 420 U/mg in the supernatant was measured, representing a 22.7-fold purification. The quality of product was indicated by the RZ (Reinheiszahl) value, which is an absorbance ratio of A403/A275, a measure of hemin content. The RZ value was increased from 0.19 for soybean extract to 0.75 after affinity precipitation. Compared to specifications of Sigma product, the SBP from the affinity precipitation has a specific activity up to 7 fold higher and a RZ value higher than the specified minimum (Table 3).

TABLE 3
Characterization of crude and purified SBP samples

| | Total protein | Purification | Specific | |
|---|---|---|---|---|
| | (mg) | Fold | Activity (U/mg) | RZ Value |
| Crude | 3.8 | 1 | 18.5 | 0.19 |
| Purified | 0.16 | 22.7 | 420 | 0.75 |
| Sigma | — | — | 50-150 | ≧0.5 |

The product stream was further analyzed by SDS-PAGE, which showed significantly reduced number of bands present. While a single homogenous solution was not evident, the target protein was significantly enriched in the product stream (FIG. 3), corroborating with the 22.7-fold increase in specific activity. Additionally, total carbohydrate and fucose content of purified SBP were also analyzed. After purification, there was a significant reduction of both mannose and xylose content in the product stream (Table 2). The ratio of mannose to GlcNAc was reduced from 6.5 to 3.4, close to the literature value of 3.3, which was from purified SBP. Similarly, xylose content in isolated SBP is substantially closer to the literature value, suggesting significant enrichment of the target protein as a result of this one-step purification. Notably the fucose content in purified sample was close to the level reported for purified SBP (Gray et al 1996), suggesting that a majority of fucose present in the sample are associated with SBP (Table 2).

Taken together, the affinity precipitation described was effective to enrich the fucose-containing SBP from a mixture containing other glycoproteins. While a homogenous SBP was not evident, most impurity present in plant crude extract was removed and the product stream is significantly enriched with SBP, which should meet the requirements for most common applications of SBP such as research reagent.

Ligand Reuse.

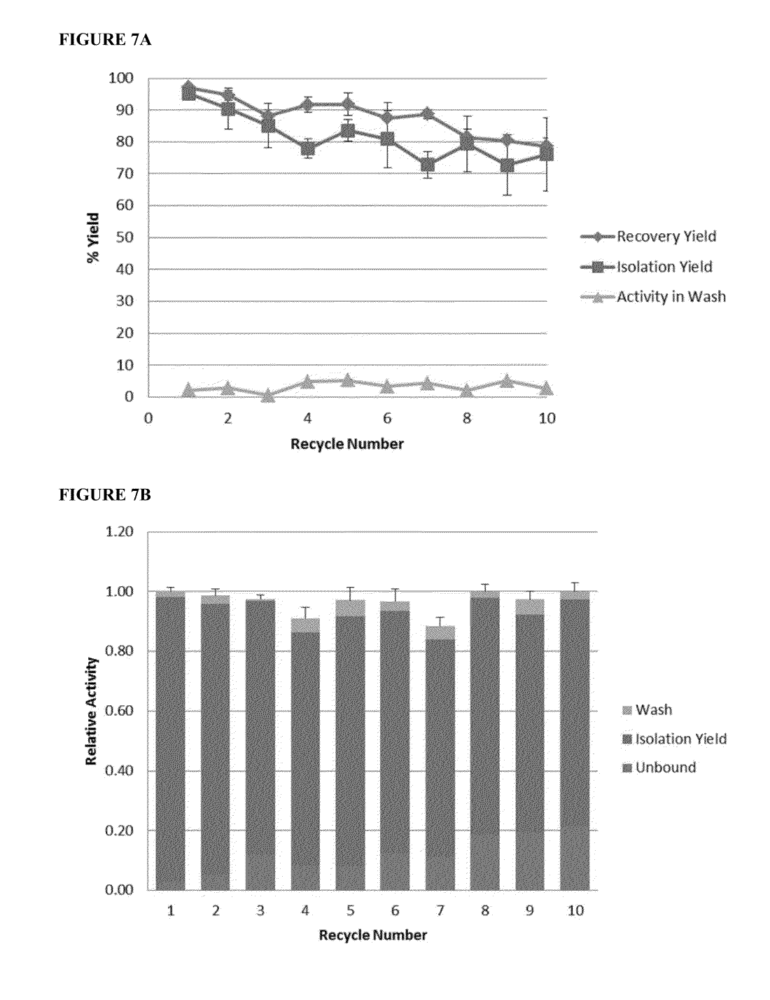

As affinity ligand is possibly the most important cost contributor in an affinity precipitation process, we investigated the possibility of its reuse. To this end, after each purification, the ligand was isolated from SBP by temperature-induced precipitation. Before the ligand could be used for the next cycle purification, it is necessary to regenerate ligand by releasing fucose bound to the ligand. Therefore, a wash step was added to the recycle procedure. The pelleted ligand was resuspended in 100 uL PBS solution and resolubilized, releasing residual fucose from the ligand. The wash solution was subject to ITC again to pellet the ligand, which was used in the next cycle of purification with fresh crude. The resulting wash fluid was analyzed for trace activity, designated as “wash” in FIGS. 7A and 7B. This additional step restored the ligand function for additional rounds of recycle via dilution and releasing the cognate sugar from the complex.

FIG. 7A details the purification results of the SBP each time the RSL-ELP was recycled. Recovery yield represents the SBP activity captured by the ligand and present in the complex with the RSL-ELP after precipitation. This yield was decreased gradually from >95% to about 80%. The isolation yield represents the SBP activity after its elution from the complex and removal of ligand from the elution solution via ITC. Similar to the recovery yield, the isolation yield also showed an appreciable decrease over the 10 recycle of ligand, from initial 95% to 75%. The loss in wash step seemed to relatively minor, <5%.

FIG. 7B illustrates the distribution of all measured activity throughout the purification process. This unbound activity was measured from the crude after purification with the affinity ligand, and represents the SBP in the crude solution not captured by the ligand. The total combined enzyme activity from three fractions was close to 100%, suggesting that no activity is lost due to denaturation or inactivation. Apparently, activity present in the “unbound” fraction was responsible to the decreasing isolation yield as the number of ligand reuse increased. Despite noticeable decline of ligand efficiency, the isolation yield averages at 81.4% over the course of 10 recycles.

These results showed that the affinity ligand can be reused for 10 times as long as a wash step to remove residual fucose is added between two purifications. The ability to reuse ligand is a unique advantage in the design of the affinity precipitation.

An interesting result from Example 1 indicates that RSL-ELP forms a trimer, just like naked RSL (Kostlanova et al 2005). As such, according to the structural study by Kostlanova et al, there are two fucose binding sites per lectin. Theoretically, only one fucose binding to the ligand is needed for capture of a SBP molecule, which corresponds to a molar ratio of ligand to SBP, 0.5:1. However, this theoretic ratio gave only 20% recovery (FIG. 6). This could be explained by steric hindrance that prevents a single lectin molecule from binding two fucose sugars on two separate SBP molecules. Increasing the ratio to 1:1, however, improved the recovery only to 50%, indicating the binding of lectin to target protein was not as simple as one ligand:one protein as the ratio would suggest. About 95.7+/−0.5% recovery yield could be achieved using a ratio of 3:1, representing two molar excess for the ligand with respect to target glycoprotein. Since on average there are 5.6 fucose moieties per SBP (Gray et al 1996), this ratio corresponds to the saturation state that all fucose present on the SBP are bound to lectin. Increasing the ratio beyond saturation is not expected to increase recovery and this observation is consistent with this expectation.

This study suggests that fucose on the same protein molecule binds preferentially. This notion is supported by studies by other researchers (Mammen et al 1998; Wang and Amin 2014). Accordingly, the ratio that gives a near complete recovery is the ratio that gives binding saturation. Therefore, the ratio for full recovery could be predicted by the number of cognate sugar on a protein. This understanding is useful as it greatly simplifies optimizations of an affinity precipitation.

As two molar excess of ligand is needed for full recovery and ligand is likely the most expensive component in the process, a possibility of ligand reuse was investigated as a way to reduce its cost impact. This requires a method to regenerate the ligand after each cycle. This was achieved by a wash step using a pH buffer containing no fucose. This simple wash process allowed the ligand to regain much of the binding ability, and very easy to implement, compared to other regeneration methods (Sheth et al 2014). However, a gradual decline of isolation yield was observed as number of reuse increases. Despite the decline, it was demonstrated that the ligand could be used for 10 times, giving an averaged 80% isolation yield based on initial input of soybean peroxidase.

This ligand recyclability is one feature of this approach, significantly different from other approaches that require direct association of ELP and the target (Banki et al 2005; Fong et al 2009). Further analysis showed that the decrease of isolation yield was due to decline of binding capacity of the ligand as the number of recycle increases. Repetitive temperature fluctuations may have caused denaturation of the lectin after multiple recycles, which could be mitigated by using a low temperature for precipitation by including salt (FIGS. 7A and 7B). An alternative explanation for the decline of binding capacity is the incomplete dissociation of fucose from ligand in the wash step, which may suggest to increase the volume of wash buffer or add a second wash step to helo dissociation.

### Materials and Methods

Chemicals.

All chemicals were purchased from Sigma-Aldrich Chemical Co. (St. Louis, Mo.) unless otherwise specified and were used without further purification. Soybeans were purchased from a local farmers market.

Fusion Protein Expression and Fractionation.

The lectin from Ralstonia solanacearum (RSL) was transcriptionally fused to an ELP peptide. This fusion protein (RSL-ELP40) was expressed in E. coli as described above. A single colony of E. coli UT5600 transformant was selected and cultured in 3 mL LB broth and ampicillin overnight at 37° C. and 250 rpm. This seed culture was used to inoculate LB broth culture containing ampicillin to an OD600 of 0.1. This larger culture was grown at 37° C. and 250 rpm to a cell density of OD600 of about 0.4, at which point isopropyl-β-D-thio-galactoside (IPTG) was added to the culture to a final concentration of 1 mM to induce the production of the fusion protein. The cultivation temperature was then reduced to 18° C. and the cells were grown for an additional 48 hours.

Periplasmic and cytoplasmic fractions were prepared according to the method described in the pETsystem manual (EMD Chemicals, San Diego, Calif.). A culture was harvested by centrifugation. The pellet was re-suspended in phosphate buffered saline (PBS) solution, sonicated, and centrifuged at 6,000 g at 4° C. for 20 min. The supernatant was saved as a cytoplasmic fraction.

Soybean Crude Protein Preparation.

Soybeans were soaked in 10% (w/v) in 0.1 M potassium phosphate buffer (pH 7.0) at room temperature overnight. The hydrated hull was removed from the bean, and the isolated hulls were again soaked in the same buffer for overnight, and then homogenized by vortexing for 2 minutes. The mixture was centrifuged for 20 minutes at 4° C. at 13,000×g. The supernatant was collected and concentrated about 20 times by acetone precipitation.

Monosaccharide Characterization.

To analyze the crude carbohydrate composition, the glycans are first hydrolyzed. The hydrolysis is achieved by addition of 6 N HCl to the protein mixture and heating to 121° C. for 45 minutes. After neutralizing with NaOH to a pH of 7, the sample was centrifuged for 30 seconds at 15,000 g to removed precipitates. The sample was diluted for analysis.

The released monosaccharide samples were analyzed by High Performance Anion-Exchange Chromatography (HPAEC) using a DIONEX system equipped with an ED50 electro-chemical detector (Sunnyvale, Calif.). Monosaccharides were separated on a CarboPac PA-20 column (Dionex). Detection was through pulsed amperometry (waveform: t=0.41 s, p=−2.00 V; t=0.42 s, p=−2.00 V; t=0.43 s, p=0.60 V; t=0.44 s, p=−0.10 V; t=0.50 s, p=−0.10 V). The mobile phase consisted of degassed solution A containing 100 mM sodium hydroxide and solution B containing deionized water. The mobile phase was pressurized with inert gas (He) to prevent interference of airborne carbon dioxide. An isocratic method containing 2% buffer A and 98% buffer B was pumped at a flow rate of 0.5 mL/min.

Acetone Precipitation.

Chilled acetone was added in a ratio of 5 to 1 to the crude protein solution. The mixture was vortexed, then refrigerated at −20° C. for an hour. The precipitates were collected by centrifugation at 6,000×g for 15 minutes. The acetone supernatant was removed, and the pellet was resuspended in PBS. An SBP activity assay as performed to ensure no loss of activity.

Inverse Temperature Cycling.

The transition temperature of the constructs was determined spectrophotometrically in a 96-well microplate reader (Spectramax M5, Molecular Devices, Sunnyvale, Calif.). Each well contained 200 mL of PBS with 0, 0.5, or 1.0M NaCl with ligand fusion at a concentration of 5 mg/mL. Temperature was increased (in 2° C. increments) from 30 to 50° C. and the absorbance at 310 nm was measured.

SBP Activity Assay.

For peroxidase assay, a solution of 0.0017M hydrogen peroxide was prepared by diluting 1 mL of 30% hydrogen peroxide to 100 mL with DI water. One milliliter of this solution was further diluted into 50 mL of 0.2M potassium phosphate buffer pH 7.0. A 0.0025M 4-aminoantipyrine solution was prepared in 0.17M phenol solution. The hydrogen peroxide solution and aminoantipyrine solution were mixed 1:1 ratio, and diluted horseradish peroxidase was added. Absorbance of the resulting mixture was measured at 510 nm over a period of 5 min. at 25° C. in a microplate reader, Spectramax M5 (Molecular Devices). Specific activity is calculated as follows:

$$Units \text{/} mg = \frac{\frac{Δ {Abs}_{510}}{min}}{6.58 \times \frac{mg enzyme}{mL reaction mixture}}$$

SDS-PAGE.

Each sample was combined in a 1:1 ratio with SDS sample buffer (10% SDS, 10% b-mercaptoethanol, 0.3M Tris-HCl (pH 6.8), 0.05% bromophenol blue, 50% glycerol), boiled for 5 minutes, and resolved by 12.5% (w/v) SDS-PAGE. Each gel was detected by Coomassie blue staining (Bio-Rad).

### Example 3

### Saialic Acid Binding Lectin Fusion Construct for Glycoprotein Purification by Affinity Precipitation

In an effort to expand the platform of the successful lectin-ELP fusion constructs, a new sialic acid binding lectin is produced as a fusion construct for the purpose of affinity precipitation purification.

Construction of VCNA-ELP40 Ligand.

This Vibrio cholera neuraminidase (VCNA) is comprised of three (3) distinct domains: a neuraminidase catalytic domain, and two flanking lectin regions (Crennell et al., 1994). Of these two lectin domains, the N-terminal lectin has been found to bind with high affinity to sialic acid (Kd of 30 μM) and similar affinity to sialic acid containing substrates, α-2,3-sialyllactose and α-2,6-sialyllactose (Moustafa et al., 2004). This 21 kD binding site is efficient in targeting terminal sialic acid moieties and does not require the addition of metal ions for binding. In the same manner as previous work with a fucose binding lectin, this lectin domain of the gene was C-terminally fused to a glycine rich linker connecting the gene encoding the (VPGVG)₄₀ sequence of the ELP domain (Arnold & Chen, 2014; also the above Example 1). In addition to these functional regions, an N-terminal TAT signal sequence was included so as to allow for secretion and easy isolation of the fusion construct after expression in E. coli. This signal sequence, when used in tandem with an E. coli mutant carrying a deletion of the lpp encoded lipoproteins, results in membrane permeability, allowing the translocated protein to leak into the media (Shin & Chen, 2008). The fusion ligand was termed VCNA-ELP40.

Production of Ligand.

Initially, a cultivation temperature of 18° C. was used, as this was the primary temperature used for expression. There was concern that too high a cultivation temperature may cause the ELP in vivo to fold and precipitate, facilitating early inclusion bodies that are not translocated. Two different IPTG concentrations were used to induce, and the total protein and recombinant protein levels were monitored at 24 and 48 hours. The results are tabulated in Table 4 below.

TABLE 4
Protein levels at 18° C. cultivation

| | |
|---|---|
| 0.1 mM | 1.0 mM |
| IPTG | IPTG |

A. Total Protein (mg/L)

| | | |
|---|---|---|
| 24 hours | 370 ± 38 | 220 ± 19 |
| 48 hours | 510 ± 52 | 330 ± 30 |

B. VCNA-ELP40 (mg/L)

| | | |
|---|---|---|
| 24 hours | 80 ± 8 | 40 ± 4 |
| 48 hours | 100 ± 10 | 70 ± 7 |

Other culture temperatures were tested as well (data not shown). However, increasing temperature had a detrimental effect on protein expression. It was concluded that a 48 hour, 18° C. cultivation after induction with 0.1 mM IPTG produced the best production levels of the VCNA-ELP40 fusion construct in E609Y.

By utilizing this secretion pathway, homogenization steps are avoided, and one-step isolation of the product is possible by simple temperature cycling. This precipitates the construct out of the culture media to be used for a purification scheme.

Characterization.

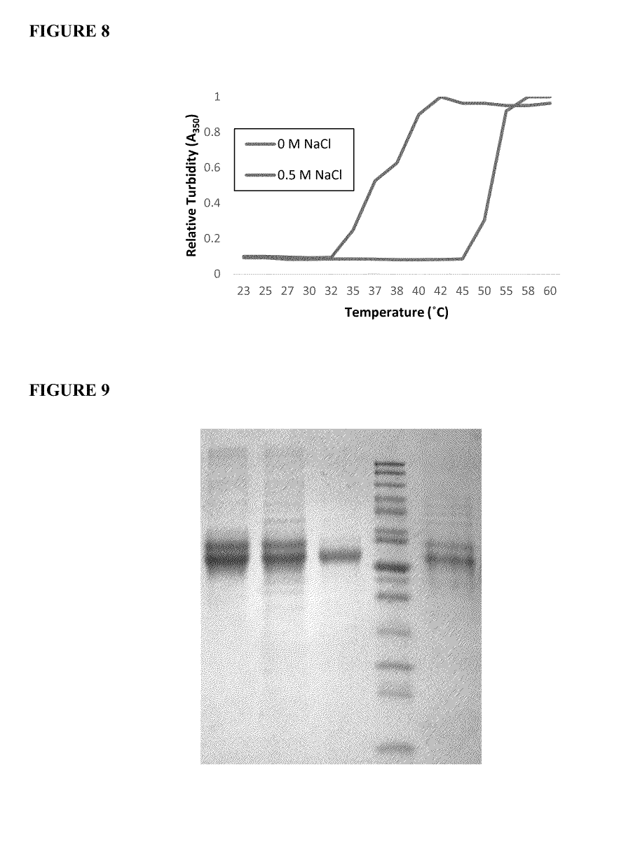

The VCNA-ELP40 fusion construct was tested for retained functionality. First, the ELP domain, which confers inverse temperature solubility to the ligand, was monitored by observing turbidity as a function of temperature, as illustrated in FIG. 8. The transition temperature was calculated at 50% of the max turbidity, which in this fusion case was about 52° C. However, addition of salt still worked as an effective additive, and lowered the transition temperature to about 37° C.

The lectin domain was tested by targeting a model glycoprotein and a negative control. The model glycoprotein used here was fetuin, a sialylated protein. VCNA-ELP40 was added to a fetuin solution in a 2:1 molar ratio of lectin to glycoprotein. The predetermined ideal ITC method of 1M salt and transition temperature of 37° C. was used to isolate the fusion protein and the bound target, and the protein was eluted from the lectin using 2 mM sialic acid solution. If the lectin is functional, an isolate of the fetuin should be attainable. As seen below in the SDS-PAGE in FIG. 9, analysis of the purification, fetuin was isolated. Not all the fetuin was removed from solution; the ratio of 2:1 was not sufficient in targeting all the desired glycoprotein. But the initial purification of fetuin ensures the VCNA lectin domain remains functional after fusion to the ELP domain.

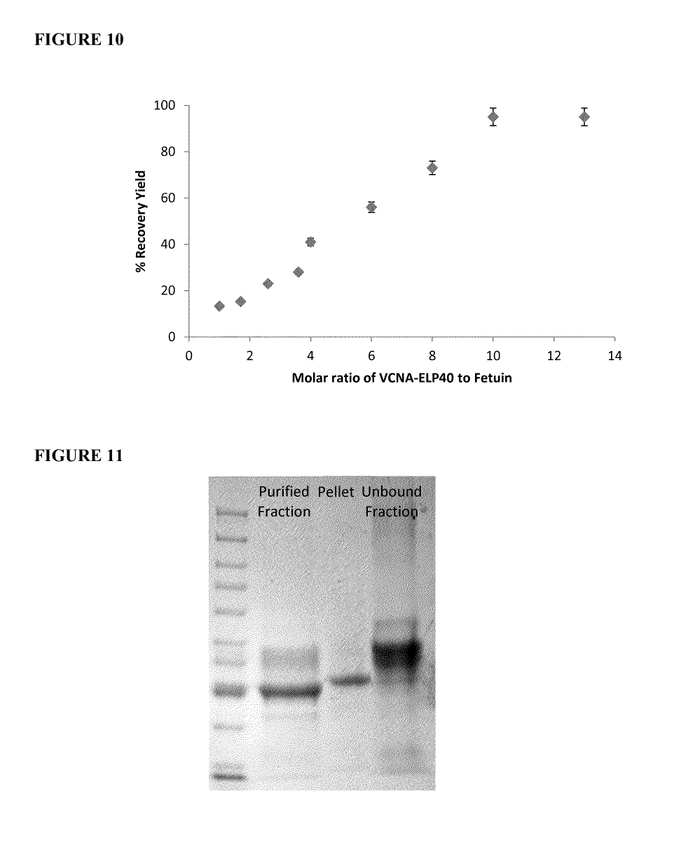

The VCNA binding portion does not form multimers, so with only one binding site per lectin, the ratio required was higher than previous RSL-ELP40 experiments. Also, fetuin has, on average, about 13.6 sialic acid residues per molecule, which is also more than the previous fucose content of HRP experiments (Spiro & Bhoyroo, 1974; Wuhrer et al., 2005). Because the ratio is related to complete titration of lectin to target sites, the molar ratio for complete capture would be around 13:1 VCNA to fetuin. To get a full profile, several ratios leading up to 13 were tested and observed. As seen in the graph below in FIG. 10, complete capture occurred around 10:1 VCNA to fetuin.

Fetuin Isolated from CHO Media.

The affinity precipitation purification technique was tested on crude CHO cell media by spiking with the desire amount of target model glycoprotein. The purpose of this was three-fold. First, a balanced ratio based on previous studies was used to ensure no loss of capture efficiency in the CHO cell media solution. Secondly, the purified product was monitored to confirm no high abundance of competing glycoprotein contaminants was targeted. Finally, other large bands in the crude were scrutinized to observe depletion of other significant contaminants.

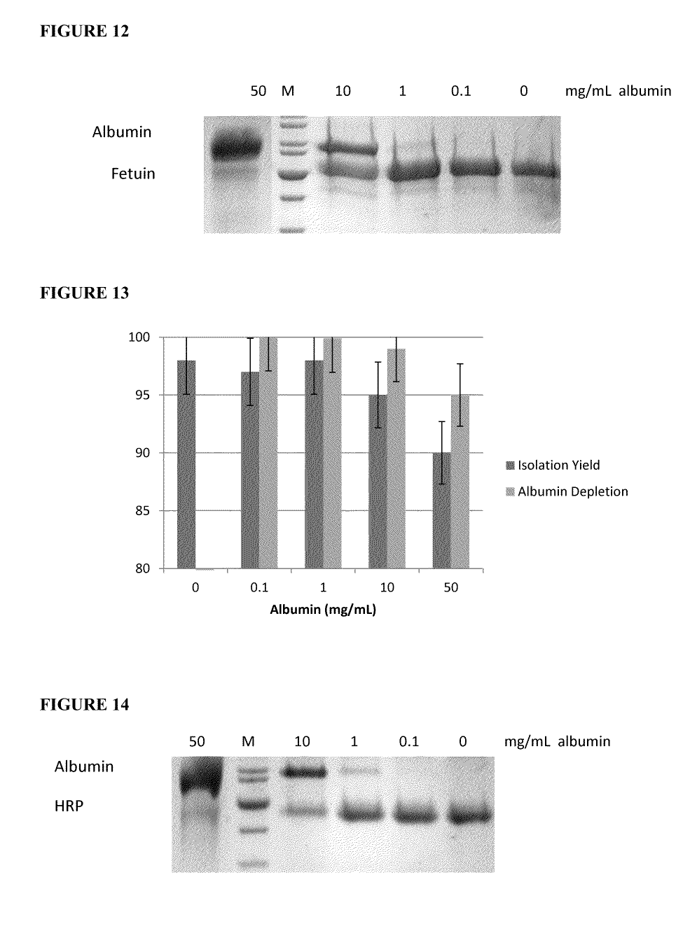

First, the VCNA-ELP40 construct was used to target a fetuin spiked media sample. 100 μL of extracellular CHO growth media was spiked with 18 μg of fetuin. VCNA-ELP40 was added in a ratio of 10:1 to the fetuin, and performed ITC using the VCNA-ELP40 favored conditions (37° C.+0.5 M NaCl). The elution of the fetuin from the ligand was done with an elution buffer containing 2 mM SA in PBS, and the ligand was isolated with another round of ITC. The SDS-PAGE of the fractions is shown below in FIG. 12.

As depicted in the SDS-PAGE in FIG. 11, the VCNA-ELP40 was successfully isolated using the ITC and elution methods. No remaining fetuin is visible in the ELP ligand lane. The unbound fraction is still predominantly albumin, with some variation of other contaminants seen at other molecular weights as well. The purified fraction, which ideally would contain only fetuin, still shows the presence of some albumin. Based off protein quantification, about 60±3% of the fetuin was isolated. The estimated purity in the SDS-PAGE is about 70%. The amount of remaining albumin in the product stream was measured as well. The reduction of this abundant albumin ranges from 94-97% reduction from the original media. This is a promising way to reduce highly abundant proteins.

VCNA Performance in High Albumin.

Over abundant glycoprotein depletion is an important step in many purification stages, specifically in the area of diagnostics. Albumin in blood samples range from 34-54 g/L, and is the dominant protein, often obscuring the investigation into other lower abundant proteins.

In order to simulate this complication, over abundant protein depletion studies were performed with fetuin, VCNA-ELP40, and a variable amount of bovine serum albumin in solution. This purification was done in 5 solutions of varying albumin content containing 0, 0.1, 1, 10, and 50 mg/mL albumin respectively. This range spans from typical culture concentrations up to the excipient concentrations to seek a point of optimal or failure of purification performance. A consistent ratio of 10:1 VCNA ligand to fetuin was used. ITC was performed by the addition of 0.5 M NaCl and heating to 37° C., and elution was done with 2 mM sialic acid in PBS. The final purified forms are depicted via SDS-PAGE analysis below in FIG. 12.

Results are summarized in FIG. 13, illustrating some loss of isolation yield with increasing albumin content. However, the albumin depletion remained fairly high at about 95% depletion even in the highest case.

RSL Performance in High Albumin.

Using RSL-ELP40, the HRP activity could be monitored to determine a more quantitative isolation yield in the presence of excess albumin. A ratio of 12:1 of RSL:HRP was added to the high albumin solution, as this is a sufficient amount to target the majority of the HRP. ITC was performed at 45° C., and eluted with 1 mM fucose, per the originally defined optimal conditions. The final purified forms from 1 round of ITC are depicted via SDS-PAGE analysis below in FIG. 14.

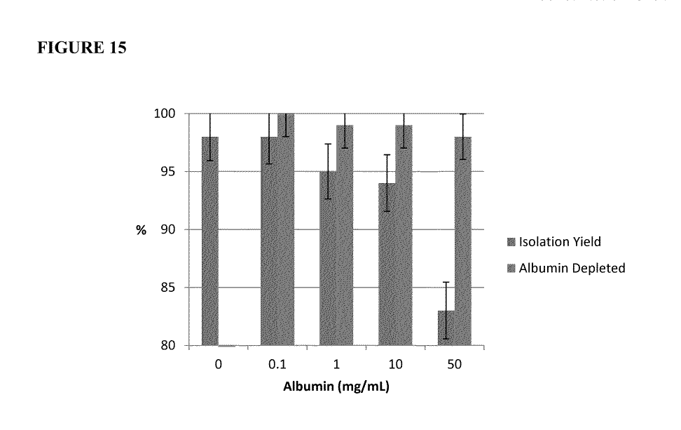

Apparently, a visible amount of residual albumin was still present as low as with the 1 mg/mL albumin solution, however it was greatly depleted. To further inspect the purification of the HRP, the activity of this solution was measured, and is reported below. An estimate of reduced albumin is reported concurrently in FIG. 15 to illustrate depletion and enrichment performance.

### Materials and Methods

Materials.

All chemicals were purchased from Sigma-Aldrich Chemical Co. (St. Louis, Mo.) unless otherwise specified and were used without further purification.

Bacterial Strains and Plasmids.

Escherichia coli strain K12 UT5600 (F-ara-14 leuB6 secA6 lacY1 proC14 tsx-67 Δ(ompT-fepC)266 entA403 trpE38 rfbD1 rpsL109 xyl-5 mtl-1 thi-1) was used as a host for all cloning steps. Plasmid pQTAT containing an N-terminal TAT signal sequence was used as the expression vector (Shin et al, 2011). The artificial gene, a 10 amino acid glycine rich linker and 20 VPGVG repeats as Example 1) was synthesized by GenScript. The ELP segment with 20 repeats of pentapeptide was oligomerized to crete 40 and 60 pentamer ELP, respectively, which was subsequently fused with the same linker and lectin in the same manner as the first construct. The synthetic genes and the expression vector pQTH, were digested with BamHI and HindIII restriction enzymes (NEB), and ligated to form the respective plasmids for fusion expression.

Fusion Expression.

This fusion proteins (VCNA-ELP40 and RSL-ELP40) were expressed in E. coli as described in Example 1. A single colony of E. coli 609Y transformant was selected and cultured in 3 mL LB broth and ampicillin overnight at 37° C. and 250 RPM. This seed culture was used to inoculate 100 mL LB broth culture containing ampicillin to an OD600 of 0.1. This larger culture was grown at 37° C. and 250 RPM to a cell density of OD600 of about 0.4, at which point isopropyl-β-D-thio-galactoside (IPTG) was added to the culture to a final concentration of 1 mM to induce the production of the fusion protein. The cultivation temperature was then reduced to 18° C. and the cells were grown for an additional 48 hours.

The extracellular fraction was collected as the supernatant after pelleting the cells by centrifugation at 6,000 g at 4° C. for 25 min. This was repeated until supernatant was clear. Periplasmic and cytoplasmic fractions were prepared according to the method described in the pET system manual (EMD Chemicals, San Diego, Calif.).

Heat Shock Induction.

Cultures were grown first at 37° C. after seeding. Upon reaching an optical density of 0.5-0.6 at OD600, the flasks were subjected to a heat shock of 42° C. for 20 minutes. The cultures were then removed and allowed to cool to room temperature for 10 minutes before the addition of IPTG to initiate protein expression. The cultivation temperature was then reduced to 30° C. and the cells were grown for an additional 48 hours.

Isolation with Inverse Temperature Cycling (ITC).

The solution was heated to the transition temperature of the ELP in an Eppendorf Thermomixer for 5 minutes at 300 RPM. The solution was then centrifuged at 16,000×g for 1 minute. The supernatant was removed, and the pellet resuspended in elution buffer. The resuspended solution was chilled at 4° C. for 30 minutes to maximize resolubilization of the ELP constructs. This chilled solution was centrifuged again for 5 minutes at 16,000×g for 5 minutes to remove unspecific precipitates, and the supernatant was collected as the purified protein fraction.

Turbidity Measurements.

The transition temperature of the constructs was determined spectrophotometrically in a 96-well microplate reader (Spectramax M5, Molecular Devices, Sunnyvale, Calif.). Each well contained 200 mL of PBS with 0 or 0.5M NaCl with ligand fusion at a concentration of 5 mg/mL. Temperature was increased (in 2° C. increments) from 22° C. to 60° C. and the absorbance at 310 nm was measured.

SDS-PAGE.

Each sample was combined in a 1:1 ratio with SDS sample buffer (10% SDS, 10% β-mercaptoethanol, 0.3M Tris-HCl (pH 6.8), 0.05% bromophenol blue, 50% glycerol), boiled for 5 minutes, and resolved by 12.5% (w/v) SDS-PAGE. Each gel was detected by Coomassie blue staining (Bio-Rad).

Protein Quantification.

The soluble protein concentration was measured using the Bradford assay with Bio-Rad reagent (Bio-Rad, Hercules, USA) and bovine serum albumin as a standard.

### REFERENCES

Arnold, L. & Chen, R. (2014) Novel thermo-responsive fucose binding ligands for glycoprotein purification by affinity precipitation. Biotechnol Bioeng. 111: 413-417.
Banki M R, Feng L A, Wood D W. 2005. Simple bioseparations using self-cleaving elastin-like polypeptide tags. Nature Methods 2:659-661.
Chattopadhyay K, Mazumdar S (2000) Structural and Conformational Stability of Horseradish Peroxidase: Effect of Temperature and pH. Biochemistry 39:263-270. doi: 10.1021/bi990729.
Chen R R. 2007. Permeability issues in whole-cell bioprocesses and cellular membrane engineering. Applied microbiology and biotechnology 74:730-8.
Chow D C, Dreher M R, Trabbic-Carlson K, Chilkoti A. 2006. Ultra-high expression of a thermally responsive recombinant fusion protein in E. coli. Biotechnology progress 22:638-46.
Christensen T, Hassouneh W, Trabbic-carlson K, and chikoti A, Predicting transition temperatures of elastin-like polypeptide fusion proteins, Biomacromolecules, 2013, 14(5): 1514-9.
Christensen T, Amiram M, Dagher S, Trabbic-Carlson K, Shamji M F, Setton L a, Chilkoti A. 2009. Fusion order controls expression level and activity of elastin-like polypeptide fusion proteins. Protein science: a publication of the Protein Society 18:1377-87.
Conley A J, Joensuu J J, Jevnikar A M, Menassa R, Brandle J E. 2009. Optimization of elastin-like polypeptide fusions for expression and purification of recombinant proteins in plants. Biotechnology and bioengineering 103:562-73.
Crennell, S., Garman, E., Laver, G., et al. (1994) Crystal structure of Vibrio cholerae neuraminidase reveals dual lectin-like domains in addition to the catalytic domain. Structure 2: 535-44.
Egrie, J. C., et al. (1993) S7. 7 The role of carbohydrate on the biological activity of erythropoietin. Glycoconjugate Journal 10.4: 263-263.
Ekins S, Xu J J (2009) Drug Efficacy, Safety, and Biologics Discovery: Emerging Technologies and Tools. 336.
Floss D M, Schallau K, Rose-John S, Conrad U, Scheller J. 2010. Elastin-like polypeptides revolutionize recombinant protein expression and their biomedical application. Trends in biotechnology 28:37-45.
Fong B, Wu W, Wood D W (2009) Optimization of ELP-intein mediated protein purification by salt substitution. Protein Expr Purif 66:198-202.
Gabius H-J, André S, Jimenez-Barbero J, Romero A, Solis D (2011) From lectin structure to functional glycomics: principles of the sugar code. Trends Biochem Sci 36:298-313.
Ge X, Yang D S C, Trabbic-carlson K, Kim B, Chilkoti A, Filipe C D M, Jhe-M S W, Ls O, Engineering B (2005) Self-Cleavable Stimulus Responsive Tags for Protein Purification without Chromatography. 11228-11229.
Ghazarian H, Idoni B, Oppenheimer S B (2011) A glycobiology review: carbohydrates, lectins and implications in cancer therapeutics. Acta Histochem 113:236-47. doi: 10.1016/j.acthis. 2010.02.004
Gillikin J W, Graham J S (1991) Purification and Developmental Analysis of the Major Anionic Peroxidase from the Seed Coat of Glycine max. Plant Physiol 96:214-20.
Gottschalk B U (2010) Downstream Processing of Monoclonal Antibodies: from High Dilution to High Purity. BioPharmInternational.com 1-17.
Gray J S, Yang B Y, Hull S R, Venzke D P, Montgomery R (1996) The glycans of soybean peroxidase. Glycobiology 6:23-32.
Hailu G, Weersink A, Cahlik F (2010) Examining the Prospects for Commercialization of Soybean Peroxidase. AgBioForum 13:263-273.
Henriksen A, Mirza 0, Indiani C, Teilum K, Smulevich G, Welinder K G, Gajhede M (2001) Structure of soybean seed coat peroxidase: A plant peroxidase with unusual stability and haem-apoprotein interactions. 108-115.
Husain Q (2009) Peroxidase mediated decolorization and remediation of wastewater containing industrial dyes: a review. Rev Environ Sci Bio/Technology 9:117-140. doi: 10.1007/s11157-009-9184-9.
Jacobs, K., Shoemaker, C., Rudersdorf, R., et al. (1985) Isolation and Characterization of Genomic and cDNA Clones of Human Erythropoietin. Nature 313: 806-810.
Jefferis R (2009) Glycosylation as a strategy to improve antibody-based therapeutics. Nat Rev Drug Discov 8:226-34. doi: 10.1038/nrd2804
Jelkmann, W. (2004) Molecular biology of erythropoietin. Intern. Med. 43: 649-59.
Jungbauer, A. (1993) Review Preparative chromatography of biomolecules. J. Chromatogr. 639: 3-16.
Kamal J K A, Behere D V (2002) Thermal and Conformational Stability of Seed Coat Soybean Peroxidase. Biochemistry 41:9034-9042.
Kobayashi Y, Tateno H, Ogawa H, Yamamoto K, Hirabayashi J. (2014) Comprehensive list of lectins: origins, natures, and carbohydrate specificities. Methods Mol Biol.; 1200:555-77. doi: 10.1007/978-1-4939-1292-645.
Kostlanova N, Mitchell E P, Lortat-Jacob H, Oscarson S, Lahmann M, Gilboa-Garber N, Chambat G, Wimmerova M, Imberty A. 2005. The fucose-binding lectin from Ralstonia solanacearum—A new type of beta-propeller architecture formed by oligomerization and interacting with fucoside, fucosyllactose, and plant xyloglucan. Journal of Biological Chemistry 280:27839-27849.
Lam S K, Ng T B (2011) Lectins: production and practical applications. Appl Microbiol Biotechnol 89:45-55. doi: 10.1007/s00253-010-2892-9
Lee L Y, Hincapie M, Packer N, Baker M S, Hancock W S, Fanayan S (2012) An optimized approach for enrichment of glycoproteins from cell culture lysates using native multi-lectin affinity chromatography. J Sep Sci 35:2445-52. doi: 10.1002/j ssc.201200049
Lin M, Rose-John S, Grötzinger J, Conrad U, Scheller J. 2006. Functional expression of a biologically active fragment of soluble gp130 as an ELP-fusion protein in transgenic plants: purification via inverse transition cycling. The Biochemical journal 398:577-83.
Linné-Larsson E, Galaev I, Lindahl L, Mattiasson B (1996) Affinity precipitation of Concanavalin A with p-aminophenyl-α-D-glucopyranoside modified Eudragit S-100. Bioseparation 6:273-282.
Liu F, Tsai S-L, Madan B, Chen W (2012) Engineering a high-affinity scaffold for non-chromatographic protein purification via intein-mediated cleavage. Biotechnol Bioeng 109:2829-35. doi: 10.1002/bit.24545
Madan B, Chaudhary G, Cramer S M, Chen W. 2013. ELP-z and ELP-zz capturing scaffolds for the purification of immunoglobulins by affinity precipitation. Journal of biotechnology 163:10-6.
Mammen M, Choi S-K, Whitesides G M (1998) Polyvalent Interactions in Biological Systems: Implications for Design and Use of Multivalent Ligands and Inhibitors. Angew Chemie Int Ed 37:2754-2794. doi: 10.1002/(SICI)1521-3773(19981102)37:20<2754:: AID-ANIE2754>3.3.CO;2-V
McDaniel J R, Radford D C, Chikoti A, A unified model for denovodesign of elastin-like polypeptides with tunable inverse transition temperatures, Biomacromolecules, 2013, 14(8): 2866-72.
Mceldoon J P, Dordick J S (1996) Unusual Thermal Stability of Soybean Peroxidase. Biotechnol Prog 7938:555-558.
Meyer D E, Chilkoti a. 1999. Purification of recombinant proteins by fusion with thermally-responsive polypeptides. Nature biotechnology 17:1112-5.
Meyer D E, Chilkoti A. Quantification of the effects of chain length and concentration on the thermal behavior of elastin-like polypeptides. Biomacromolecules. 2004; 5:846-851. [PubMed: 15132671]
Meyer D E, Trabbic-Carlson K, Chilkoti a. 2001. Protein purification by fusion with an environmentally responsive elastin-like polypeptide: effect of polypeptide length on the purification of thioredoxin. Biotechnology progress 17:720-8.
Misaizu, T., Matsuki, S., Strickland, T. W., et al. (1995) Role of antennary structure of N-linked sugar chains in renal handling of recombinant human erythropoietin. Blood 86: 4097-104.
Moustafa, I., Connaris, H., Taylor, M., et al. (2004) Sialic acid recognition by Vibrio cholerae neuraminidase. J. Biol. Chem. 279: 40819-26.
Nakano M, Kondo A, Kakehi K, Taniguchi N (2006) Glycomics—a new target for pharmaceuticals. Drug Discov Today Technol 3:39-47. doi: 10.1016/j.ddtec.2006.03.005
Patchornik G, Albeck A (2005) Free nonimmobilized ligands as a tool for purification of proteins. Bioconjug Chem 16:1310-5.
Raman R, Venkataraman M, Ramakrishnan S, Lang W, Raguram S, Sasisekharan R (2006) Advancing glycomics: implementation strategies at the consortium for functional glycomics. Glycobiology 16:82R-90R. doi: 10.1093/glycob/cwj080
Sakharov I Y, Berlina A N, Zherdev A V, Dzantiev B B (2010) Advantages of soybean peroxidase over horseradish peroxidase as the enzyme label in chemiluminescent enzyme-linked immunosorbent assay of sulfamethoxypyridazine. J Agric Food Chem 58:3284-9. doi: 10.1021/jf904338f
Sheth R D, Jin M, Bhut B V, Li Z, Chen W, Cramer S M (2014) Affinity precipitation of a monoclonal antibody from an industrial harvest feedstock using an ELP-Z stimuli responsive biopolymer. Biotechnol Bioeng 111:1595-603. doi: 10.1002/bit.25230
Shimazu M, Mulchandani A, Chen W. 2003. Thermally triggered purification and immobilization of elastin-OPH fusions. Biotechnology and bioengineering 81:74-9.
Shin H-D, Chen R R. 2008. Extracellular recombinant protein production from an Escherichia coli lpp deletion mutant. Biotechnology and bioengineering 101:1288-96.
Simnick A J, Lim D W, Chow D, Chilkoti A. 2007. Biomedical and biotechnological applications of elastin-like polypeptides. Polymer Reviews 47:121-154.
Skibeli, V. (2001) Sugar profiling proves that human serum erythropoietin differs from recombinant human erythropoietin. Blood 98: 3626-3634.
Spiro R G. 1960. Studies on fetuin, a glycoprotein of fetal serum. Journal of Biological Chemistry 235:2860-2869.
Spiro, R. G. & Bhoyroo, V. D. (1974) Structure of the O-Glycosidically Linked Carbohydrate Units of Fetuin Structure of the 0-Glvcosidicallv of Fetuin*Carbohydrate. 5704-5717.
Taniguchi N, Ekuni A, Ko J H, Miyoshi E, Ikeda Y, Ihara Y, Nishikawa A, Honke K (2001) Review A glycomic approach to the identification and characterization of glycoprotein function in cells transfected with glycosyltransferase genes. 239-247.
Tian L, Sun S S M. 2011. A cost-effective ELP-intein coupling system for recombinant protein purification from plant production platform. PloS one 6:e24183.
VanHaandel M J, Saraber F C, Boersma M G, Laane C, Fleming Y, Weenen H, Rietjens I M (2000) Characterization of different commercial soybean peroxidase preparations and use of the enzyme for N-demethylation of methyl N-methylanthranilate To produce the food flavor methylanthranilate. J Agric Food Chem 48:1949-54.
Varki A, Cummings, Esko (2009) Essentials of Glycobiology, 2nd edn. Cold Spring Harbor Laboratory Press, Cold Spring Harbor, N.Y.
Wafa Hassouneh, Trine Christensen, and Ashutosh Chilkoti (2010) Elastin-like Polypeptides as a Purification Tag for Recombinant Proteins. Curr Protoc Protein Sci. August; CHAPTER: Unit-6.11. doi:10.1002/0471140864.ps0611s61.
Wang L-X, Amin M N (2014) Chemical and chemoenzymatic synthesis of glycoproteins for deciphering functions. Chem Biol 21:51-66. doi: 10.1016/j.chembiol.2014.01.001
Wood D W (2010) Non-Chromatographic Recombinant Protein Purification by Self-Cleaving Purification Tags. Sep Sci Technol 45:2345-2357. doi: 10.1080/01496395.2010.507665
Wuhrer M, Balog C I a, Koeleman C a M, Deelder A M, Hokke C H. 2005. New features of site-specific horseradish peroxidase (HRP) glycosylation uncovered by nano-LC-MS with repeated ion-isolation/fragmentation cycles. Biochimica et biophysica acta 1723:229-39.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. An affinity binding ligand comprising a fusion construct comprising lectin fused with a thermal responsive elastin-like protein (ELP), wherein said affinity binding ligand is for purifying a glycoprotein by affinity precipitation.

2. The affinity binding ligand of claim 1, wherein said lectin derived from bacteria Ralstonia solanacearum (RSL) binds to fucose.

3. The affinity binding ligand of claim 1, wherein said lectin derived from Vibrio cholera neuraminidase (VCNA) binds to saialic acid.

4. The affinity binding ligand of claim 1, wherein said ELP comprises 20, 40, or 60 ELP pentapeptide repeats of VPGVG (SEQ ID NOS 3-5, respectively).

5. The affinity binding ligand of claim 1, further comprising a 10 amino acid glycine rich linker (GGGGS)₂ (SEQ ID NO: 2).

6. The affinity binding ligand of claim 1, wherein said fusion construct is RSL-ELP20 comprising lectin derived from RSL fused with 20 VPGVG (SEQ ID NO:3) ELP repeats.

7. The affinity binding ligand of claim 1, wherein said fusion construct is RSL-ELP40 comprising lectin derived from RSL fused with 40 VPGVG (SEQ ID NO:4) ELP repeats.

8. The affinity binding ligand of claim 1, wherein said fusion construct is RSL-ELP60 comprising lectin derived from RSL fused with 60 VPGVG (SEQ ID NO:5) ELP repeats.

9. The affinity binding ligand of claim 1, wherein said fusion construct is VCNA-ELP20 comprising lectin derived from VCNA fused with 20 VPGVG (SEQ ID NO:3) ELP repeats.

10. The affinity binding ligand of claim 1, wherein said fusion construct is VCNA-ELP40 comprising lectin derived from VCNA fused with 40 VPGVG (SEQ ID NO:4) ELP repeats.

11. The affinity binding ligand of claim 1, wherein said fusion construct is VCNA-ELP60 comprising lectin derived from VCNA fused with 60 VPGVG (SEQ ID NO:5) ELP repeats.

12. The affinity binding ligand of claim 1, wherein said glycoprotein is a plant peroxidase.

13. The affinity binding ligand of claim 12, wherein said plant peroxidase is horseradish peroxidase (HRP) or soybean peroxidase (SBP).

14. The affinity binding ligand of claim 1, wherein said glycoprotein is a sialylated protein.

15. The affinity binding ligand of claim 14, wherein said sialylated protein is fetuin.

16. A method for purifying a glycoprotein of interest by affinity precipitation comprising:
a) constructing a fusion binding ligand comprising lectin fused with ELP pentapeptide repeats of VPGVG (SEQ ID NO:1);
b) contacting the fusion binding ligand with said glycoprotein of interest; and
c) isolating and purifying said glycoprotein of interest by affinity precipitation.

17. The method of claim 16, wherein said lectin derived from bacteria binds to fucose, saialic acid, or a glycan portion of said glycoprotein.

18. The method of claim 17, wherein said glycoprotein is a fucosylated or a sialylated protein.

19. The method of claim 16, wherein said glycoprotein of interest is isolated and purified with Inverse Temperature Cycling (ITC).


SEQUENCE LISTING (6 sequences)

SEQ ID NO: 1 - protein, 5 aa, Artificial Sequence
  Description of Artificial Sequence Synthetic peptide
VPGVG

SEQ ID NO: 2 - protein, 10 aa, Artificial Sequence
  Description of Artificial Sequence Synthetic peptide
GGGGSGGGGS

SEQ ID NO: 3 - protein, 100 aa, Artificial Sequence
  Description of Artificial Sequence Synthetic polypeptide
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 4 - protein, 200 aa, Artificial Sequence
  Description of Artificial Sequence Synthetic polypeptide
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 5 - protein, 300 aa, Artificial Sequence
  Description of Artificial Sequence Synthetic polypeptide
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 6 - protein, 1650 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG